(19) United States
(12) Patent Application Publication
(10) Pub. No.: US 2025/0025360 A1
(43) Pub. Date: Jan. 23, 2025

(54) MOBILE NEGATIVE PRESSURE CHAMBER

(71) Applicants: SOONCHUNHYANG UNIVERSITY INDUSTRY ACADEMY COOPERATION FOUNDATION, Asan-si (KR); KOREA ADVANCED INSTITUTE OF SCIENCE AND TECHNOLOGY, Daejeon (KR)

(72) Inventors: Seong Jun CHOI, Seoul (KR); Jung Wan PARK, Hwaseong-si (KR); Su Ha HAN, Bucheon-si (KR); Hyung Soo KIM, Daejeon (KR); Hae Won PARK, Daejeon (KR); Sung Jin PARK, Daejeon (KR); Ji Hye PARK, Daejeon (KR)

(73) Assignees: SOONCHUNHYANG UNIVERSITY INDUSTRY ACADEMY COOPERATION FOUNDATION, Asan-si (KR); KOREA ADVANCED INSTITUTE OF SCIENCE AND TECHNOLOGY, Daejeon (KR)

(21) Appl. No.: 18/496,095

(22) Filed: Oct. 27, 2023

(30) Foreign Application Priority Data
Jul. 18, 2023 (KR) 10-2023-0092802

(51) Int. Cl. A61G 10/00; A61G 10/02

(52) U.S. Cl. CPC A61G 10/005; A61G 10/023

(57) ABSTRACT

Disclosed is a mobile negative pressure chamber, that includes a bed, a main cover separably coupled to the bed, and having a main cavity, an auxiliary cover coupled to one side of the bed to be separable, that seals the main cavity while being engaged with the main cover, and having an auxiliary cavity, in which a lower body of the surgical recipient is located in an interior thereof, a main treatment part provided in at least one of the main cover and the auxiliary cover, and provided with a glove mounted on an arm of the surgical operator, and an auxiliary treatment part provided in at least one of the main cover and the auxiliary cover, and into and from which the arm of the surgical operator or a medical instrument is inserted and extracted.

### CROSS-REFERENCE TO RELATED APPLICATIONS

A claim for priority under 35 U.S.C. § 119 is made to Korean Patent Application No. 10-2023-0092802 filed on Jul. 18, 2023 in the Korean Intellectual Property Office, the entire contents of which are hereby incorporated by reference.

### BACKGROUND

Embodiments of the inventive concept described herein relate to a mobile negative pressure chamber.

Problems are arising due to the unexpected spread of infectious diseases in medical processes related to the acceptance and treatment of patients. From SARS in 2002, H1N1 influenza A in 2009, Middle East Respiratory Syndrome (MERS) in 2015, and COVID-19 in 2019, various new infectious diseases are occurring and spreading rapidly, and as the number of infected people increases explosively, problems such as lack of hospital rooms are emerging. In particular, a problem of isolation of infected people, which is a characteristic of infectious diseases, is a problem that has to be solved to prevent the spread of infectious diseases and prevent the loss of medical resources that may occur during the treatment process.

Recently, in the case of COVID-19, which is becoming a global pandemic, it is a respiratory infectious disease that has an airborne transmission route through droplets, and the indirect propagation route through surfaces contaminated with droplets is also effective, and a possibility of propagation through aerosols can also be ruled out and so it is known to have an incredibly high infectiousness.

To effectively isolate a patient with infectious diseases, a negative pressure ward, of which a pressure of an interior is lower than an outside, is necessary. Negative pressure rooms require facilities for pressure control, and thus, they usually exist as separate wards and may not be equipped in sufficient numbers within the hospital.

For this reason, conventionally, in emergency cases, a technology was used to temporarily construct an entire negative pressure ward outdoors. However, even in wards, medical staffs are still exposed to infectious agents. Accordingly, medical staffs perform medical procedures while wearing various protective gear, and these difficulties still arise in overall medical procedures in multiple hospital beds. In addition, there is a limit in temporarily installing an entire negative pressure hospital room, and thus, there is still a shortage of negative pressure isolation beds.

Meanwhile, sealed negative pressure devices for transport are also used to transport infected people. However, a conventional negative pressure device for transport can transport a patient in isolation, but it is difficult to treat and diagnose the patient during transport.

Accordingly, the conventional negative pressure device for transport require hospitalization of the patient in an isolation room or negative pressure room after the patient arrived at a medical institution, due to a possibility of spreading the patient's respiratory infectious disease. Accordingly, the treatment and diagnosis of the patient may be delayed.

### PRIOR TECHNOLOGICAL DOCUMENTS

### Patent Documents

(Patent document 1) Korean Patent Application Publication No. 10-2022-0075909 (2022.06.08.)

### SUMMARY

Embodiments of the inventive concept provide a mobile negative pressure chamber which may allow a surgical operator to promptly treat and diagnose a surgical recipient with no danger of propagation of infectious diseases of a respiratory system of the surgical recipient while the surgical recipient is transported in isolation.

The problems to be solved by the inventive concept are not limited to the above-mentioned problems, and the other unmentioned problems will be clearly understood from the following description by an ordinary person.

According to an embodiment, a mobile negative pressure chamber includes a bed, a main cover coupled to one side of the bed to be separable, and having a main cavity, in which an upper body of a surgical recipient is located in an interior thereof, an auxiliary cover coupled to one side of the bed to be separable, that seals the main cavity while being engaged with the main cover, and having an auxiliary cavity, in which a lower body of the surgical recipient is located in an interior thereof, a main treatment part provided in at least one of the main cover and the auxiliary cover, and provided with a glove mounted on an arm of the surgical operator, and an auxiliary treatment part provided in at least one of the main cover and the auxiliary cover, and into and from which the arm of the surgical operator or a medical instrument is inserted and extracted.

Furthermore, the main treatment part may include a glove port formed in at least one of the main cover and the auxiliary cover, and a glove valve that opens and closes the glove port, and by which the glove is elastically supported.

Furthermore, the glove valve may include a glove frame coupled to the glove port to be separable, a glove holder coupled to the glove frame to be separable, and having a holder recess, by which one end of the glove is elastically supported, on an outer peripheral surface thereof, and a glove cap coupled to the glove frame to be separable, and that opens and closes the glove port.

Furthermore, the main treatment part may further include an elastic member surrounding one end of the glove elastically supported by the outer peripheral surface of the glove holder, and that generates an elastic force that attaches the one end of the glove to the glove holder.

Furthermore, an attachment recess, into which the one end of the glove attached by the elastic member is inserted, may be formed on the outer peripheral surface of the glove holder.

Furthermore, the auxiliary treatment part may include an insertion/extraction port formed in at least one of the main cover and the auxiliary cover, and to and from which the arm of the surgical operator or the medical instrument is inserted and extracted, an insertion/extraction valve that opens and closes the insertion/extraction port, and a sealing member that seals the insertion/extraction port, and that seals the insertion/extraction port while being attached to the arm of the surgical operator or the medical instrument when the arm of the surgical operator or the medical instrument is inserted into and extracted from the insertion/extraction port.

Furthermore, the insertion/extraction valve may include an insertion/extraction frame coupled to the insertion/extraction port to be separable, an insertion/extraction holder stacked on the insertion/extraction frame, an insertion/extraction coupling part coupling the insertion/extraction frame and the insertion/extraction holder such that insertion/extraction frame and the insertion/extraction holder are separable, and an insertion/extraction cap coupled to the insertion/extraction frame to be separable, and that opens and closes the insertion/extraction port, and the sealing member may be interposed and held between the insertion/extraction frame and the insertion/extraction holder.

Furthermore, the sealing member may include a first sealing film disposed on an outer surface of the insertion/extraction frame, and having an annular shape, a pair of second sealing films laminated on opposite sides of the first sealing film, and each of which has an arc shape, and a pair of third sealing films laminated on the pair of second sealing films, respectively, disposed on an inner surface of the insertion/extraction holder, and each of which has a fan shape, and the pair of third sealing films may be disposed to overlap each other to seal the insertion/extraction port, and seals the insertion/extraction port while being attached to the arm of the surgical operator or the medical instrument when the arm of the surgical operator or the medical instrument is inserted into and extracted from the insertion/extraction port.

Other detailed items of the inventive concept are included in the detailed description of the inventive concept and the drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating a mobile negative pressure chamber according to an embodiment of the inventive concept;

FIG. 2 is an exploded perspective view illustrating a mobile negative pressure chamber according to an embodiment of the inventive concept;

FIG. 3 is a perspective view illustrating a state, in which an auxiliary cover is separated from a mobile negative pressure chamber, according to an embodiment of the inventive concept;

FIG. 4 is an enlarged view illustrating a fixing frame of a mobile negative pressure chamber according to an embodiment of the inventive concept;

FIG. 5 is an exploded perspective view illustrating a main treatment part of a mobile negative pressure chamber according to an embodiment of the inventive concept;

FIG. 6 is a cross-sectional view illustrating a mobile negative pressure chamber according to an embodiment of the inventive concept;

FIG. 7 is an exploded perspective view illustrating an auxiliary treatment part of a mobile negative pressure chamber according to an embodiment of the inventive concept;

FIG. 8 is a perspective view illustrating a sealing member of a mobile negative pressure chamber according to an embodiment of the inventive concept; and

FIG. 9 is a cross-sectional view illustrating an auxiliary treatment part of a mobile negative pressure chamber according to an embodiment of the inventive concept.

### DETAILED DESCRIPTION

The above and other aspects, features, and advantages of the inventive concept will become apparent from the following description of the following embodiments given in conjunction with the accompanying drawings. However, the inventive concept is not limited by the embodiments disclosed herein but will be realized in various different forms, and the embodiments are provided only to make the disclosure of the inventive concept complete and fully inform the scope of the inventive concept to an ordinary person in the art, to which the inventive concept pertains, and the inventive concept will be defined by the scope of the claims.

The terms used herein are provided to describe the embodiments but not to limit the inventive concept. In the specification, the singular forms include plural forms unless particularly mentioned. The terms “comprises” and/or “comprising” used herein does not exclude presence or addition of one or more other elements, in addition to the aforementioned elements. Throughout the specification, the same reference numerals denote the same elements, and “and/or” includes the respective elements and all combinations of the elements. Although “first”, “second” and the like are used to describe various elements, the elements are not limited by the terms. The terms are used simply to distinguish one element from other elements. Accordingly, it is apparent that a first element mentioned in the following may be a second element without departing from the spirit of the inventive concept.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, embodiments of the inventive concept will be described in detail with reference to the accompanying drawings.

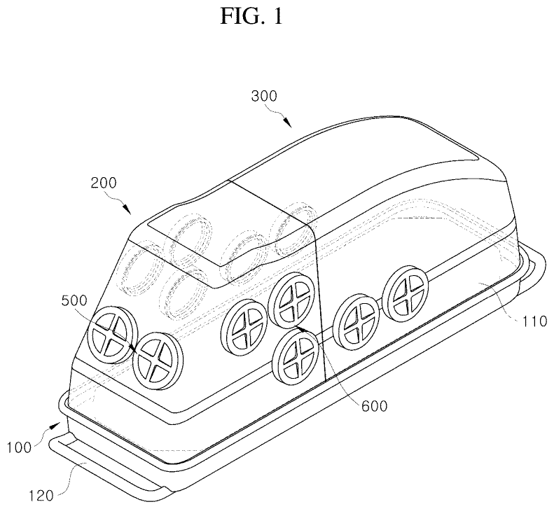

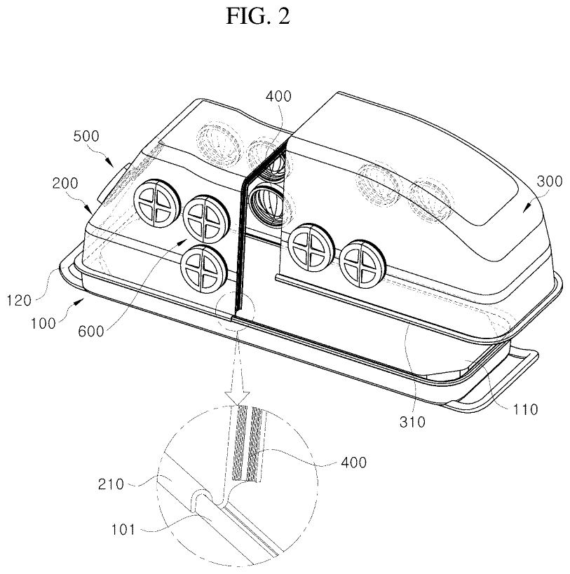

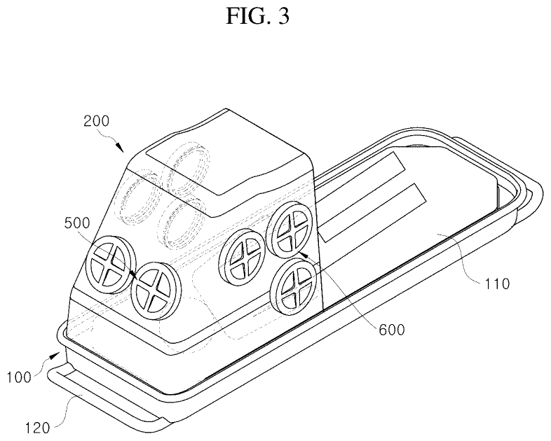

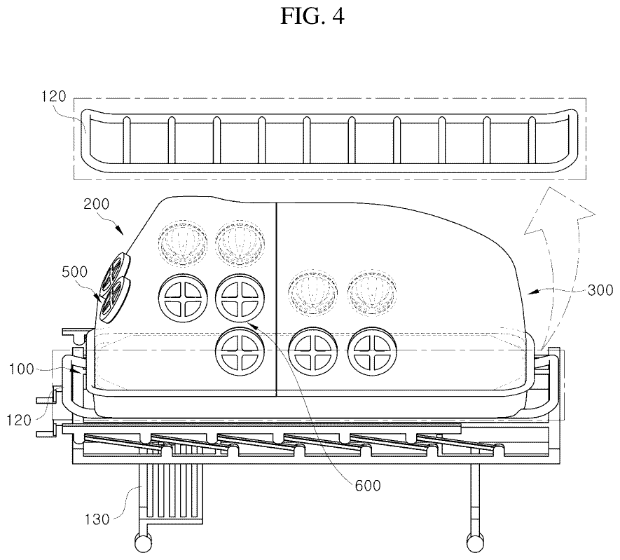

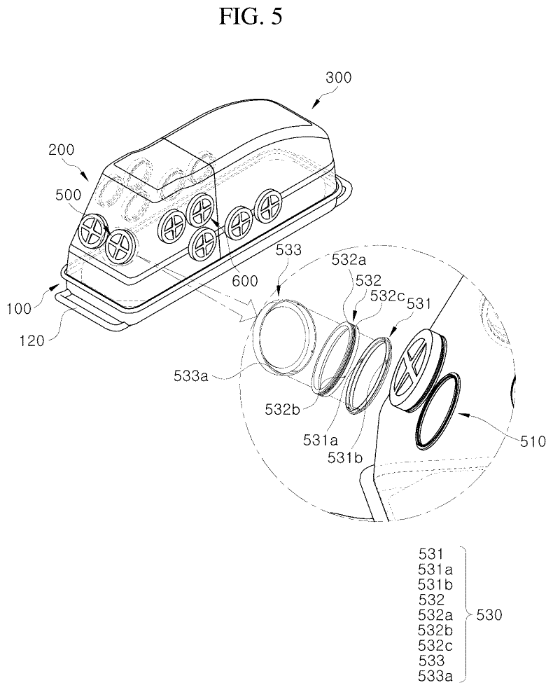

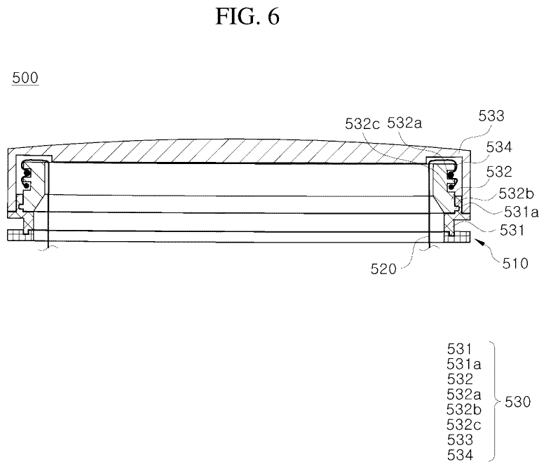

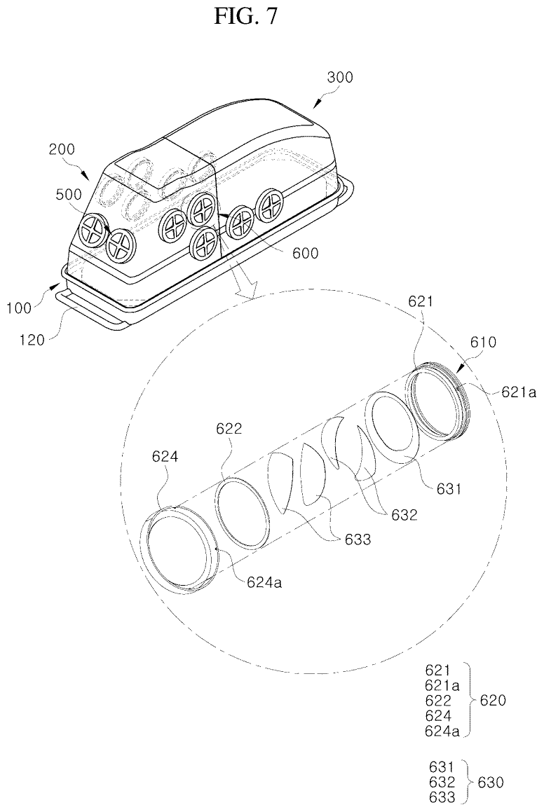

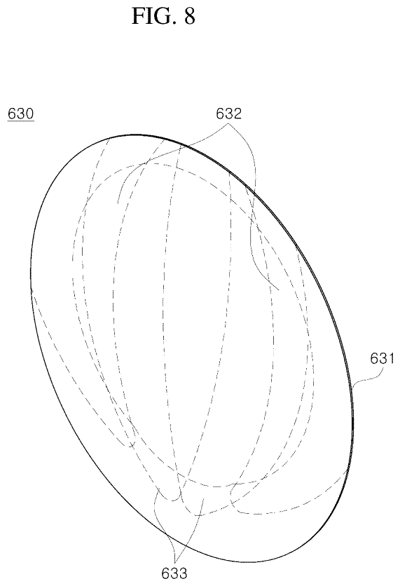

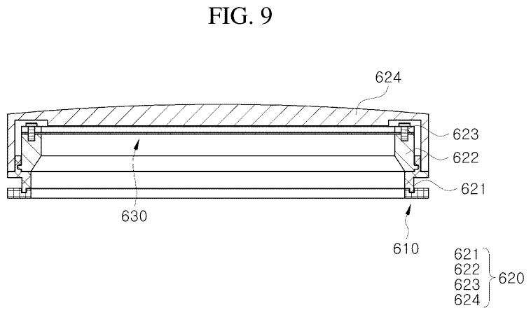

FIG. 1 is a perspective view illustrating a mobile negative pressure chamber according to an embodiment of the inventive concept, FIG. 2 is an exploded perspective view illustrating the mobile negative pressure chamber according to an embodiment of the inventive concept, FIG. 3 is a perspective view illustrating a state, in which an auxiliary cover is separated from the mobile negative pressure chamber, according to an embodiment of the inventive concept, FIG. 4 is an enlarged view illustrating a fixing frame of the mobile negative pressure chamber according to an embodiment of the inventive concept, FIG. 5 is an exploded perspective view illustrating a main treatment part of the mobile negative pressure chamber according to an embodiment of the inventive concept, FIG. 6 is a cross-sectional view illustrating the mobile negative pressure chamber according to an embodiment of the inventive concept, FIG. 7 is an exploded perspective view illustrating an auxiliary treatment part of the mobile negative pressure chamber according to an embodiment of the inventive concept, FIG. 8 is a perspective view illustrating a sealing member of the mobile negative pressure chamber according to an embodiment of the inventive concept, and FIG. 9 is a cross-sectional view illustrating the auxiliary treatment part of the mobile negative pressure chamber according to an embodiment of the inventive concept.

As illustrated in FIGS. 1 to 9, the mobile negative pressure chamber according to an embodiment of the inventive concept may include a bed 100, a main cover 200, an auxiliary cover 300, a main treatment part 500, and an auxiliary treatment part 600. Hereinafter, for convenience of description, a medical staff will be defined as an operator, and a patient with respiratory infectious diseases will be defined as a surgical recipient.

The bed 100 functions to a seat for the surgical recipient. As an example, a cushion 110 for absorbing an impact that is applied to the surgical recipient while the surgical recipient is transported may be provided on an upper surface of the bed 100. As another example, referring to FIG. 4, a fixing frame 120 for fixing a couch 130 may be provided on a lower surface of the bed 100. The fixing frame 120 may have a lattice form to be easily fixed to the couch 130 for transporting the surgical recipient.

The main cover 200 functions to cover an upper body of the surgical recipient seated on the bed 100. The main cover 200 may be coupled to a front side of the bed 100 to be separable, and may have a main cavity, in which the upper body of the surgical recipient is located, in an interior thereof. Here, the main cavity may be maintained in a negative pressure state as air is suctioned by a negative pressure pump (not illustrated).

The auxiliary cover 300 functions to cover a lower body of the surgical recipient seated on the bed 100. The auxiliary cover 300 may be coupled to a rear side of the bed 100 to be separable, and may have an auxiliary cavity, in which the lower body of the surgical recipient is located in an interior thereof. Here, the auxiliary cavity may be maintained in a negative pressure state as air is suctioned by a negative pressure pump (not illustrated).

Meanwhile, a Peltier element for adjusting a temperature of the main cavity in a noncontact scheme may be provided on an outer surface of the main cover 200, and a Peltier element for adjusting a temperature of the auxiliary cavity in a noncontact scheme also may be provided on an outer surface of the auxiliary cover 300.

A guide rail 101 may be formed at a circumference of the bed 100, and a main slider 210 that is fixed to a front side of the guide rail 101 may be formed at a circumference of the main cover 200. Here, the guide rail 101 may have a circular cross-sectional shape, and the main slider 210 may have an arc-shaped cross-sectional shape that surrounds the circular cross-sectional shape.

Furthermore, an auxiliary slider 310 that is fixed to a rear side of the guide rail 101 may be formed at a circumference of the guide rail 101. Here, the guide rail 101 may have a circular cross-sectional shape, and the auxiliary slider 310 may have an arc-shaped cross-sectional shape that surrounds the circular cross-sectional shape. (see FIG. 2)

The main cover 200 may have a dual wall, in which an outer wall and an inner wall are disposed to be spaced apart from each other, and a plurality of spacers for maintaining an interval between the outer wall and the inner wall may be provided. Furthermore, the main cover 200 may be coated with a thermoplastic polyurethane elastomer.

Furthermore, the auxiliary cover 300 also may have a dual wall, in which an outer wall and an inner wall are disposed to be spaced apart from each other, and a plurality of spacers for maintaining an interval between the outer wall and the inner wall may be provided. Furthermore, the auxiliary cover 300 may be coated with a thermoplastic polyurethane elastomer.

A rear side of the main cover 200 may be opened. Here, the opened rear side of the main cover 200 may be covered by a gasket 400. As an example, the gasket 400 may include a silicon material.

Furthermore, a front side of the auxiliary cover 300 may be opened. Here, the opened front side of the auxiliary cover 300 may be covered by the gasket 400. As an example, the gasket 400 may include a silicon material.

Referring to FIGS. 5 and 6, the main treatment part 500 may be provided in at least one of the main cover 200 and the auxiliary cover 300, and a glove 520, on which an arm of the surgical operator is laid, may be provided. The main treatment part 500 may include a glove port 510 and a glove valve 530.

The glove port 510 may be formed in at least one of the main cover 200 and the auxiliary cover 300. As an example, a plurality of glove ports 510 may be provided, and the plurality of glove ports 510 may be formed in the main cover 200 and the auxiliary cover 300 while having an interval. As another example, the plurality of glove ports 510 may be formed while having an interval only in the main cover 200. As another example, a pair of glove ports 510 may be provided, and the pair of glove ports 510 may be formed in the main cover 200 while having an interval.

The glove valve 530 may open and close the glove ports 510, and the glove 520 may be elastically supported. The glove valve 530 may include a glove frame 531, a glove holder 532, a glove cap 533, and an elastic member 534.

The glove frame 531 may be coupled to the glove port 510 to be separable. For example, the glove frame 531 may be screw-coupled to the glove port 510. Furthermore, the glove frame 531 may have an annular shape.

The glove holder 532 may be coupled to the glove frame 531 to be separable, and a holder recess 532c, by which one end of the glove 520 is elastically supported, may be formed on an outer peripheral surface thereof. The glove holder 532 may have an annular shape.

The glove cap 533 may be coupled to the glove frame 531 to be separable, and may open and close the glove port 510. As an example, the glove cap 533 may have a cylindrical shape.

The elastic member 534 may generate an elastic force that attaches one end of the glove 520 to the glove holder 532 while surrounding one of a probe that is elastically supported by an outer peripheral surface of the glove holder 532. Accordingly, since the one end of the glove 520 is attached to the glove holder 532 by the elastic member 534, the one end of the glove 520 may be prevented from being separated from the glove holder 532. As an example, the elastic member 534 may have an annular shape.

An attachment recess 532a, into which the one end of the glove 520 attached by the elastic member 534 is inserted, may be formed on an outer peripheral surface of the glove holder 532. As an example, the attachment recess 532a may have an annular shape.

In an embodiment, a first stopper recess 531a may be formed on an inner peripheral surface of the glove frame 531, and a holder boss 532b that is stopped by and fixed to the first stopper recess 531a may be formed on an outer peripheral surface of the glove holder 532. Here, the first stopper recess 531a may have a first insertion area that is formed along a direction that is parallel to a central axis of the glove frame 531, and a first stopper area that is formed along a circumferential area of the glove frame 531. Furthermore, the holder boss 532b may be rotated along a circumferential direction of the glove frame 531 to be stopped by and fixed to the first stopper area of the first stopper recess 531a after being moved in a direction that is parallel to the central axis of the glove frame 531 and being inserted into the first insertion area of the first stopper recess 531a.

In an embodiment, a second stopper recess 531b may be formed on an outer peripheral surface of the glove frame 531, and a cap boss 533a that is stopped by and fixed to the second stopper recess 531b may be formed on an inner peripheral surface of the glove cap 533. Here, the second stopper recess 531b may have a second insertion area that is formed along a direction that is parallel to a central axis of the glove frame 531, and a second stopper area that is formed along a circumferential area of the glove frame 531. Furthermore, the cap boss 533a may be rotated along the circumferential direction of the glove frame 531 to be stopped by and fixed to the second stopper area of the second stopper recess 531b after being moved in a direction that is parallel to the central axis of the glove frame 531 and being inserted into the second insertion area of the second stopper recess 531b.

Referring to FIGS. 7 to 9, the auxiliary treatment part 600 may be provided in at least one of the main cover 200 and the auxiliary cover 300, and the arm of the surgical operator or the medical instrument may be inserted thereinto and extracted therefrom. The auxiliary treatment part 600 may include an insertion/extraction port 610, an insertion/extraction valve 620, and a sealing member 630.

The insertion/extraction port 610 may be formed in at least one of the main cover 200 and the auxiliary cover 300, and the arm of the surgical operator or the medical instrument may be inserted thereinto and extracted therefrom. As an example, a plurality of insertion/extraction ports 610 may be provided, and the plurality of insertion/extraction ports 610 may be formed in the main cover 200 and the auxiliary cover 300 while having an interval. As another example, the plurality of insertion/extraction ports 610 may be formed while having an interval only in the main cover 200. As another example, a pair of insertion/extraction ports 610 may be provided, and the pair of insertion/extraction ports 610 may be formed in the main cover 200 while having an interval.

The insertion/extraction valves 620 may function to open and close the insertion/extraction ports 610. The insertion/extraction valve 620 may include an insertion/extraction frame 621, an insertion/extraction holder 622, an insertion/extraction coupling part 623, and an insertion/extraction cap 624.

The insertion/extraction frame 621 may be coupled to the insertion/extraction port 610 to be separable. For example, the insertion/extraction frame 621 may be screw-coupled to the insertion/extraction port 610. Furthermore, the insertion/extraction frame 621 may have an annular shape.

The insertion/extraction holder 622 may be stacked on the insertion/extraction frame 621. As an example, the insertion/extraction holder 622 may have an annular shape. In this way, in a state, in which the insertion/extraction holder 622 is stacked on the insertion/extraction frame 621, the sealing member 630 that will be described below may be interposed and held between the insertion/extraction frame 621 and the insertion/extraction holder 622.

The insertion/extraction coupling part 623 may couple the insertion/extraction frame 621 and the insertion/extraction holder 622 such that the insertion/extraction frame 621 and the insertion/extraction holder 622 are separable. For example, a bolt may be used as the insertion/extraction coupling part 623.

The insertion/extraction cap 624 may be coupled to the insertion/extraction frame 621 to be separable, and may open and close the insertion/extraction port 610. As an example, the insertion/extraction cap 624 may have a cylindrical shape.

The sealing member 630 may seal the insertion/extraction port 610, and may seal the insertion/extraction port 610 while being attached to the arm of the surgical operator or the medical instrument when the arm of the surgical operator or the medical instrument is inserted into and extracted from the insertion/extraction port 610. The sealing member 630 may include a first sealing film 631, a pair of second sealing films 632, and a pair of third sealing films 633.

The first sealing film 631 may be disposed on an outer surface of the insertion/extraction frame 621, and may have an annular shape.

The pair of second sealing films 632 may be laminated on opposite sides of the first sealing film 631, and may have an arc shape.

The pair of third sealing films 633 may be laminated on the pair of second sealing films 632, may be disposed on an inner surface of the insertion/extraction holder 622, and may have a fan shape. Here, the pair of third sealing films 633 may be disposed to overlap each other to seal the insertion/extraction port 610, and may seal the insertion/extraction port 610 while being attached to the arm of the surgical operator or the medical instrument when the arm of the surgical operator or the medical instrument is inserted into and extracted from the insertion/extraction port 610.

In an embodiment, a slip material for slip of the arm of the surgical operator or the medical instrument may be coated on inner peripheral surfaces of the first sealing films 631, the second sealing film 632, and the third sealing films 633.

In an embodiment, a recess 621a may be formed on an outer peripheral surface of the insertion/extraction frame 621, and an extension boss 624a that is stopped by and fixed to the recess 621a may be formed on an inner peripheral surface of the insertion/extraction cap 624. Here, the recess 621a may have a first recessed area that is formed along a direction that is parallel to a central axis of the insertion/extraction frame 621, and a second recessed area that is formed along a circumferential area of the insertion/extraction frame 621. Furthermore, the extension boss 624a may be rotated along a circumferential direction of the glove frame 531 to be stopped by and fixed to the second recessed area of the recess 621a after being moved in a direction that is parallel to a central axis of the insertion/extraction frame 621 and being inserted into the first recessed area of the recess 621a.

Hereinafter, a process of transporting the surgical recipient through the mobile negative pressure chamber according to an embodiment of the inventive concept will be described.

First, the auxiliary cover 300 is separated on a rear side of the bed 100. (see FIG. 2)

Next, an upper body of the surgical recipient is introduced into the main cavity of the main cover 200.

Subsequently, as the auxiliary cover 300 is coupled to the rear side of the bed 100 and the gasket 400 of the main cover 200 and the gasket 400 of the auxiliary cover 300 are attached to each other, the main cavity of the main cover 200 and the auxiliary cavity of the auxiliary cover 300 are sealed.

Thereafter, in a state, in which the fixing frame 120 is coupled to the couch 130, the surgical recipient may be transported through wheels provided in the couch 130.

Thereafter, when it is necessary to treat the surgical recipient while the surgical recipient is transported, the surgical operator may treat the surgical recipient by using the glove 520 provided in the glove port 510 after opening the glove port 510 through the glove valve 530 of the main treatment part 500. Furthermore, the surgical operator may treat the surgical recipient after opening the insertion/extraction port 610 through the insertion/extraction valve 620 of the auxiliary treatment part 600 and then introducing the arm of the surgical operator or the medical instrument through the insertion/extraction port 610. Then, when the arm of the surgical operator or the medical instrument is inserted into and extracted from the insertion/extraction port 610, the sealing member 630 may seal the insertion/extraction port 610 while being attached to the arm of the surgical operator or the medical instrument.

According to the inventive concept, the mobile negative pressure chamber according to an embodiment of the inventive concept may allow the surgical operator to promptly treat and diagnose the surgical recipient with no danger of propagation of infectious diseases of a respiratory system of the surgical recipient while the surgical recipient is transported in isolation.

The effects of the inventive concept are not limited to the above-mentioned problems, and the other unmentioned effects will be clearly understood from the following description by an ordinary person.

Although the embodiments of the inventive concept have been described with reference to the attached drawings until now, an ordinary person in the art, to which the inventive concept pertains, may understand that the inventive concept may be carried out in other detailed forms without changing the technical spirits or the essential features. Therefore, it should be understood that the above-described embodiments are exemplary in all aspects and are not restrictive.

What is claimed is:

1. A mobile negative pressure chamber comprising:
a bed;
a main cover coupled to one side of the bed to be separable, and having a main cavity, in which an upper body of a surgical recipient is located in an interior thereof;
an auxiliary cover coupled to one side of the bed to be separable, configured to seal the main cavity while being engaged with the main cover, and having an auxiliary cavity, in which a lower body of the surgical recipient is located in an interior thereof;
a main treatment part provided in at least one of the main cover and the auxiliary cover, and provided with a glove mounted on an arm of a surgical operator; and
an auxiliary treatment part provided in at least one of the main cover and the auxiliary cover, and into and from which the arm of the surgical operator or a medical instrument is inserted and extracted.

2. The mobile negative pressure chamber of claim 1, wherein the main treatment part includes:
a glove port formed in at least one of the main cover and the auxiliary cover; and
a glove valve configured to open and close the glove port, and by which the glove is elastically supported.

3. The mobile negative pressure chamber of claim 2, wherein the glove valve includes:
a glove frame coupled to the glove port to be separable;
a glove holder coupled to the glove frame to be separable, and having a holder recess, by which one end of the glove is elastically supported, on an outer peripheral surface thereof; and
a glove cap coupled to the glove frame to be separable, and configured to open and close the glove port.

4. The mobile negative pressure chamber of claim 3, wherein the main treatment part further includes:
an elastic member surrounding one end of the glove elastically supported by the outer peripheral surface of the glove holder, and configured to generate an elastic force that attaches the one end of the glove to the glove holder.

5. The mobile negative pressure chamber of claim 4, wherein an attachment recess, into which the one end of the glove attached by the elastic member is inserted, is formed on the outer peripheral surface of the glove holder.

6. The mobile negative pressure chamber of claim 1, wherein the auxiliary treatment part includes:
an insertion/extraction port formed in at least one of the main cover and the auxiliary cover, and to and from which the arm of the surgical operator or the medical instrument is inserted and extracted;
an insertion/extraction valve configured to open and close the insertion/extraction port; and
a sealing member configured to seal the insertion/extraction port, and configured to seal the insertion/extraction port while being attached to the arm of the surgical operator or the medical instrument when the arm of the surgical operator or the medical instrument is inserted into and extracted from the insertion/extraction port.

7. The mobile negative pressure chamber of claim 6, wherein the insertion/extraction valve includes:
an insertion/extraction frame coupled to the insertion/extraction port to be separable;
an insertion/extraction holder stacked on the insertion/extraction frame;
an insertion/extraction coupling part coupling the insertion/extraction frame and the insertion/extraction holder such that insertion/extraction frame and the insertion/extraction holder are separable; and
an insertion/extraction cap coupled to the insertion/extraction frame to be separable, and configured to open and close the insertion/extraction port, and
wherein the sealing member is interposed and held between the insertion/extraction frame and the insertion/extraction holder.

8. The mobile negative pressure chamber of claim 7, wherein the sealing member includes:
a first sealing film disposed on an outer surface of the insertion/extraction frame, and having an annular shape;
a pair of second sealing films laminated on opposite sides of the first sealing film, and each of which has an arc shape; and
a pair of third sealing films laminated on the pair of second sealing films, respectively, disposed on an inner surface of the insertion/extraction holder, and each of which has a fan shape, and
wherein the pair of third sealing films are disposed to overlap each other to seal the insertion/extraction port, and seals the insertion/extraction port while being attached to the arm of the surgical operator or the medical instrument when the arm of the surgical operator or the medical instrument is inserted into and extracted from the insertion/extraction port.
